(19) United States
(12) Patent Application Publication
(10) Pub. No.: US 2016/0349276 A1
(43) Pub. Date: Dec. 1, 2016

(54) Use of Sortilin as Biomarker for Affective/Mood Disorders

(71) Applicant: H. LUNDBECK A/S, Valby (DK)

(72) Inventors: Jesper Lundhede Jepsen, Århus C (DK); Anders Nykjær, Risskov (DK); Niels Peter Ole Mors, Risskov (DK); Claus Munck Petersen, Århus C (DK); Søren Dinesen Østergaard, Copenhagen N (DK); Simon Glerup, Risskov (DK)

(73) Assignee: H. Lundbeck A/S, Valby (DK)

(21) Appl. No.: 15/117,967

(22) PCT Filed: Feb. 6, 2015
(86) PCT No.: PCT/EP2015/052506

§ 371 Date: Aug. 10, 2016

(30) Foreign Application Priority Data
Feb. 12, 2014 (DK) PA201400075

(51) Int. Cl. G01N 33/68

(52) U.S. Cl. CPC G01N 33/6896; G01N 2800/304; G01N 2800/50; G01N 2800/52

(57) ABSTRACT

The present invention relates to the use of Sortilin as a biomarker. In particular, Sortilin is useful as a biomarker for affective disorders. In one aspect of the invention Sortilin may be used to aid the diagnosing and to monitor disease progression in bipolar disorder and unipolar depression by measuring Sortilin levels. The invention further provides methods for informing the choice of treatment of bipolar disorder and unipolar depression by determining the levels of Sortilin.

The present invention relates to the use of Sortilin as biomarker. In particular, Sortilin is useful as a biomarker for affective disorders. In one aspect of the invention Sortilin may be used to diagnose, or to aid the diagnosis and to monitor disease progression in bipolar disorder and unipolar depression by measuring Sortilin levels. The invention further provides methods for informing the choice of treatment of bipolar disorder and unipolar depression by determining the levels of Sortilin.

### BACKGROUND OF THE INVENTION

Bipolar disorder is a severe psychiatric condition, which has a major negative impact on those afflicted, their relatives and society as a whole (Murray, C. J. 2012, Lancet. 380, 2197-2223). The lifetime prevalence of bipolar disorder is approximately 1-2% (Merikangas, K. R. 2011, Arch. Gen. Psychiatry. 68, 241-251) and the illness is among the leading causes of disability worldwide (Murray, C. J. 2012, Lancet. 380, 2197-2223).

Bipolar disorder is characterized by episodic pathological disturbances in mood, activity and energy. According to the 10^{th} edition of the International Classification of Disease (ICD-10) (World Health Organization 1993), the diagnosis of bipolar (affective) disorder is assigned after at least two episodes of pathological mood involving both mood poles, i.e. depression+either hypomania, mania or a mixed affective episode. However, recurrent episodes of hypomania, mania and mixed affective episodes are also classified as bipolar disorder according to ICD-10. The criteria for bipolar disorder in the 4^{th} and 5^{th} edition of the Diagnostic and Statistical Manual of Mental Disorders (DSM-IV and DSM-5) (American Psychiatric Association 1994; American Psychiatric Association 2013) differ slightly from those of the ICD-10 in that a single episode of mania or mixed episode (without any depressive episodes), is also classified as bipolar disorder.

Diagnosing of bipolar disorder is complex and error-prone. Therefore, the correct diagnosis is often delayed several years, which leads to poor prognosis since early intervention and treatment is crucial for a good outcome for the patients (Drancourt, N et al, 2013, Acta Psychiatr. Scand. 127, 136-144). One of the reasons for this delay is that current diagnostics rely solely on psychiatric examination, i.e. the gathering of information through conversation with the patient/relatives, observation of psychopathology during admission/outpatient care, and neuropsychological testing. Unlike the case for virtually all “medical” diseases, there are unfortunately no qualified paraclinical tests, such as biomarker-assays, to aid doctors in the diagnosing of bipolar disorder (Singh, I and Rose, N., 2009. Biomarkers in psychiatry. Nature. 460, 202-207; Boksa, P. 2013, J. Psychiatry Neurosci. 38, 75-77).

Once diagnosed, bipolar disorder can be treated pharmacologically with mood-stabilizing agents (lithium, various anti-epileptics) or atypical antipsychotics (Yatham, L. N, et al., 2009, Bipolar Disord. 11, 225-255; American Psychiatric Association 2002; Grunze, H., et al., 2013, World J. Biol. Psychiatry. 14, 154-219; National Institute For Health And Clinical Excellence 2006; Suppes, T., et al., 2005, J. Clin. Psychiatry. 66, 870-886; Goodwin, G. M., et al., 2009, J. Psychopharmacol. 23, 346-388). However, choosing the right drug for a patient is currently based on the trial-and-error approach as no solid predictors or biomarkers for response to a specific drug have been identified and implemented in clinical practice (Rybakowski, J. K., 2013, CNS Drugs. 27, 165-173; Frey, B. N. et al., 2013, N. Z. J. Psychiatry. 47, 321-332). This is a problem, which delays optimal treatment and worsens the prognosis for people suffering from bipolar disorder.

Thus, in order to shorten the period of undiagnosed (and therefore untreated) bipolar disorder, and to prescribe the correct treatment, there is an urgent need for the development/detection of reliable biomarkers.

While the text above focuses on bipolar disorder, the exact same challenges are present in the diagnosing and treatment of unipolar depression.

By unipolar depression is referred to depressive episode(s) (code F32) or recurrent depressive disorder (code F33) as described in the 10^{th} edition of the International Classification of Disease (ICD-10) (World Health Organization 1993, which corresponds approximately to major depressive disorder as described in the 4^{th} and 5^{th} edition of the Diagnostic and Statistical Manual of Mental Disorders (DSM-IV and DSM-5) (American Psychiatric Association 1994; American Psychiatric Association 2013).

The vacuolar protein sorting 10 protein (Vps10p) domain receptor family is a group of transmembrane molecules, which are expressed in both the developing and adult brain (Willnow, T. E. et al. 2008, Nat. Rev. Neurosci. 9, 899-909). The family consists of five structurally related receptor subtypes: Sortilin, SorLA, SorCS1, SorCS2 and SorCS3 which play important roles in the regulation of neuronal function and viability (Hermey, G., et al. 2004, J. Neurochem. 88, 1470-1476; Hermey, G. 2009, Cell Mol. Life Sci. 66, 2677-2689). The genes encoding the Vps10p-domain have been linked to the development of neuropsychiatric disorders, including bipolar disorder (Christoforou, A. et al. 2011, Mol. Psychiatry. 16, 240; Ollila, H. M. et al. 2009, Mol. Psychiatry. 14, 351; Baum, A. E. et al. 2008, Mol. Psychiatry. 13, 197-207; Takata, A. et al. 2011, Psychiatry Clin. Neurosci. 65, 280-285).

The inventors of the present invention have found that Sortilin levels were significantly altered in subjects with bipolar disorder and unipolar depression. These findings entail that, in relation to bipolar disorder and unipolar depression, Sortilin may be used 1) as a diagnostic or diagnostic aid, 2) to monitor disease progression and treatment response, and 3) to inform the choice and dose of treatment/medication.

### SUMMARY OF THE INVENTION

The present invention relates to the use of Sortilin, or a fragment of Sortilin, as biomarker for bipolar disorder and unipolar depression or the predisposition towards bipolar disorder and/or unipolar depression. The present invention further provides a method to diagnose or monitor bipolar disorder and unipolar depression comprising determining the level of Sortilin, or a fragment of Sortilin, in a sample from a subject.

In another embodiment, the present invention provides a method for improving the treatment effect, for example by informing the choice and dose of treatment/medication, in a subject suffering from bipolar disorder or unipolar depression, said method comprises
a. Determining the level of Sortilin, or a fragment of Sortilin, in a sample from a subject, and
b. Adjusting the treatment regime for the subject having decreased or increased plasma or serum levels of Sortilin, or a fragment of Sortilin, relative to control levels determined in said subject or relative to a predetermined value.

A kit for detection of Sortilin levels in a sample from a subject is also provided. The kit may be an immunoassay such as an ELISA using anti-Sortilin antibodies.

### FIGURE

FIG. 1A: Levels of serum Sortilin in subjects with bipolar disorder (BD) and unipolar depression (UD) compared to controls. FIG. 1B: Levels of serum Sortilin in subjects with bipolar disorder (BD) unipolar depression (UD), and control subjects all given in relative terms to the mean value of the control group.

FIG. 2 Data showing the serum values for individual patients and relative values for individual patients relative to control.

### DETAILED DESCRIPTION OF THE INVENTION

The present invention is based on the finding that levels of sortilin, which are equal to or lower than a predetermined value, is associated with the presence of bipolar disorder and that levels of sortilin, which are equal to or higher than a predetermined value, are associated with the presence of unipolar depression. This finding can improve the diagnostic process and the treatment in relation to bipolar disorder and unipolar depression.

It is to be understood that the term “diagnose”, “diagnosed” or “diagnosing” as used in the present application is not intended to mean that the measured levels of Sortilin in a sample will be the only parameter used when diagnosing a subject. A medical doctor will use several clinical observations in determining the correct diagnosis as for example outlined in the ICD-10 and the DSM-5. However, the present invention may assist in reaching the correct diagnosis for a given subject.

The present invention thus relates to the use of Sortilin, or a fragment of Sortilin, as biomarker. The sequence of Sortilin is given in SEQ ID NO: 1, and the term “fragment” is intended to mean an immunological measurable fragment of at least 5 amino residues, such as for example at least 10, 15 or 20 amino residues in length.

The use of Sortilin as a biomarker may in one embodiment be measured by use of a kit. Such a kit could be beneficial for psychiatric hospitals, psychiatric outpatient clinics and private practicing psychiatrists or other medical staff to increase the sensitivity and specificity in the diagnosing of bipolar disorder or unipolar depression. Furthermore, the kit could be useful in monitoring the severity of the illness or the response to treatment.

In one aspect of the invention such kit could be in the form of an immunoassay by use of antibodies directed against Sortilin, or a fragment of Sortilin. Such antibodies can readily be made by the skilled person using routine methods. For example, monoclonal antibodies may be produced by the hybridoma method first described by Kohler et al., Nature 256, 495 (1975), or may be produced by recombinant DNA methods. Monoclonal antibodies may also be isolated from phage antibody libraries using the techniques described in, for example, Clackson et al., Nature 352, 624-628 (1991) and Marks et al., J. Mol. Biol. 222, 581-597 (1991). Thus, for example, monoclonal antibodies may be obtained from hybridomas prepared from murine splenic B lymphocyte cells obtained from mice immunized with Sortilin or a fragment of Sortilin together with an appropriate adjuvant such as Freunds adjuvant. The fragment of Sortilin may be conjugated to a carrier such as KLH to make it more immunogenic. Monoclonal antibodies may also be obtained from hybridomas derived from anti-body expressing cells of immunized humans or non-human mammals such as rats, rabbits, dogs, primates, etc. Anti-Sortilin antibodies are also commercially available from for example Millipore, R&D Systems and Abcam.

The immunoassay may be in the form of a competitive assay or non-competitive assay (such as a one-site competitive assay or two-site competitive assay) and use labels such as enzymes (e.g. enzyme-linked immunosorbent assays (ELISAs) using horseradish peroxidase (HRP), alkaline phosphatase (AP) or glucose oxidase), radioactive isotopes (e.g. radioimmunoassay) or fluorogenic reporters. Many references are available to provide guidance in applying the above techniques (Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987)).

In one embodiment the kit can be in the form of an ELISA coated with anti-Sortilin antibodies to which the sample is added and incubated with anti-Sortilin antibodies before labeled anti-IgG antibody is finally added. The readout may subsequently be compared to a standard curve generated, as exemplified in the Example of the present invention, in order to establish the actual concentration. However, the level of Sortilin in a sample may also be compared to a “baseline concentration” or “control level”. For example the control level may be the level of Sortilin in a sample from a subject before the subject receives the first dose of a therapy, in order to determine the treatment effect or the need to adjust the treatment dosage or treatment period, or may be used in broader terms for a representative of the normal population, excluding any subjects with anxiety or affective disorders according to SCAN interviews (World Health Organisation 1998).

The term “sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, sputum, milk, whole blood or any blood fraction, blood derivatives, blood cells, tumors, neuronal tissue, organs or any type of tissue, any sample obtained by lavage (for example of the bronchial system), and sample of in vivo cell culture constituents. In particular, the sample may be in the form of a blood sample, plasma sample or serum sample obtained from a subject suspected of having or having bipolar disorder or unipolar depression.

In another embodiment the invention provides a method for improving the treatment effect, for example by informing the choice and dose of treatment/medication, in a subject suffering from bipolar disorder or unipolar depression. This method comprises:
a. Determining the level of Sortilin, or a fragment of Sortilin, in a sample from a subject, and
b. Adjusting the treatment regimen the subject having decreased or increased plasma or serum levels of Sortilin, or a fragment of Sortilin, relative to control levels determined in said subjects or relative to a predetermined value.

One way to determine the level of Sortilin in a subject may be by use of the kit mentioned above.

The method is therefore also directed to a method or a kit as described above wherein a measured level of Sortilin equal to or below a predetermined value is indicative of:
a. The subject having bipolar disorder,
b. The subject being predisposed to bipolar disorder, or
c. The subject with bipolar disorder being in need of treatment adjustment for example in the form of a specific medication, a higher/lower dosage or a prolonged/shortened treatment period, depending on the current treatment regimen.

Also a measured level of Sortilin equal to or above a predetermined value is thus indicative of:
a. The subject not having bipolar disorder,
b. The subject not being predisposed to bipolar disorder, or
c. The subject with bipolar disorder being in need of treatment adjustment for example in the form of a specific medication, a higher/lower treatment dosage or a prolonged/shortened treatment period, depending on the current treatment regimen.

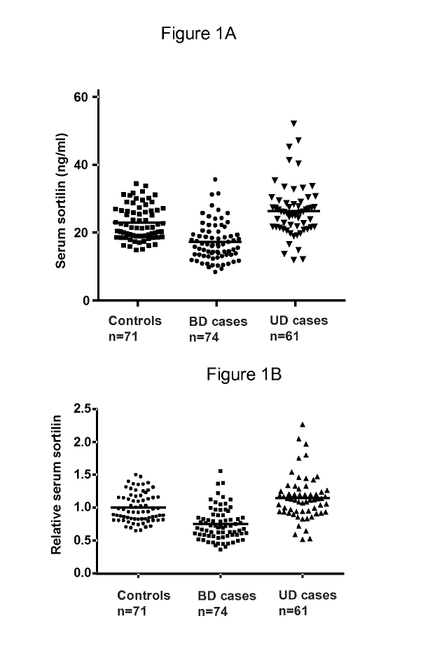

The Examples (FIG. 1A) of the present invention shows that with a serum Sortilin cutoff at approximately 18 ng/ml, corresponding to a relative value of about 0.8 (FIG. 1B), approximately 40% of subjects with bipolar disorder can be separated from the healthy controls with a high specificity and low number of false positives.

The “predetermined value” as mentioned above may thus be a Sortilin level of about 18 ng/ml, however, it is to be understood that such value has a certain confidence interval due to technical differences in handling the equipment for measurements so that about 18 ng/ml cover values within for example 16 to 22 ng/ml, such as 17 to 21 ng/ml or 18 to 20 ng/ml. The relative serum concentrations to control corresponds to about 0.4 to about 0.8, such as about 0.4 to about 0.7 or about 0.4 to about 0.6.

Many individuals with bipolar disorder are treated with lithium carbonate, anticonvulsants (e.g., valproate, carbamazepine, lamotrigine), or antipsychotics (e.g., aripiprazole, olanzapine, quetiapine or risperidone). Adjustment of these treatment regimes, e.g. by lowering/increasing the dosage, changing medicine or shortening/prolonging the treatment period, may be judged by determining the Sortilin levels in samples from the subject.

Thus in one embodiment the invention relates to a method for treating a subject suffering from bipolar disorder comprising the steps of:
a. Diagnosing the subject by the use of the above-mentioned method, or
b. Diagnosing the subject by the kit described above, and
c. Administering a therapeutically effective amount of a medication.

In another embodiment the present invention allows to differentiate between individuals with unipolar depression and healthy controls. Accordingly, a method comprising the step of determining the level of Sortilin, or a fragment of Sortilin, in a sample from a subject. The subject may be a subject suspected or diagnosed as having unipolar depression.

Because the invention provides a method for improving the treatment effect in a subject, said method comprises determining the level of Sortilin, or a fragment of Sortilin, in a sample from a subject, and adjusting the treatment regime for subjects having decreased or increased plasma or serum levels of Sortilin, or a fragment of Sortilin, relative to control levels determined in said subjects or relative to a predetermined value.

One way to determine the level of Sortilin in a subject may be by use of the above-mentioned kit. The method is therefore also directed to a method or a kit as described above wherein a measured level of Sortilin equal to or above a predetermined value is indicative of:
a. The subject having unipolar depression,
b. The subject being predisposed to unipolar depression, or
c. The subject with unipolar depression being in need of treatment adjustment for example in the form of a specific medication, a higher/lower dosage or a prolonged/shortened treatment period, depending on the current treatment regimen.

Also a measured level of Sortilin equal to or below a predetermined value is thus indicative of:
a. The subject not having unipolar depression,
b. The subject not being predisposed to unipolar depression, or
c. The subject with unipolar depression being in need of treatment adjustment for example in the form of a specific medication, a higher/lower treatment dosage or a prolonged/shortened treatment period, depending on the current treatment regimen.

The Example of the present invention shows that with a serum Sortilin cutoff at about 40 ng/ml, subjects with unipolar depression can be separated from controls with a high specificity and low number of false positives.

The “predetermined value” as mentioned above may thus be a Sortilin level of about 40 ng/ml, corresponding to a relative value of about 1.5 (FIG. 1 B), however it is to be understood that such value has a certain confidence area are around it due to technical differences in handling the equipment for measurements so that about 40 ng/ml cover values within for example 30 to 60 ng/ml, such as 35 to 50 ng/ml or 35 to 50 ng/ml. The relative serum concentrations to control corresponds to about 1.5 to about 3, such as about 1.5 to about 2.5 or about 1.5 to about 2.3.

### Experimental Details

The inventors have measured serum levels of Sortilin in subjects with bipolar disorder, unipolar depression, and controls. These experiments were carried out in collaboration with Aarhus University Hospital, Risskov (Psychiatric hospital).

Patients/Controls Used for Biomarker Identification:

The patient population consisted of 74 patients meeting ICD-10 criteria for bipolar affective disorder and the DSM-IV criteria for Bipolar I disorder (Buttenschon, H. N. et al. 2010, Psychiatr. Genet. 20, 93-101) and 61 patients meeting ICD-10 criteria for a unipolar depressive episode (Kolstad, H. A. et al. 2011, Am. J. Epidemiol. 173, 94-102; Kaerlev, L. et al. 2011, BMC Public Health. 11, 539-2458-11-539.). All patients were diagnosed by psychiatrists using the Schedules for Clinical Assessment in Neuropsychiatry (SCAN) semi-structured diagnostic interview (version 2.1) (World Health Organization 1998). The 71 controls were employees within large public service workplaces in a Danish county (Kolstad, H. A. et al. 2011, Am. J. Epidemiol. 173, 94-102; Kaerlev, L. et al. 2011, BMC Public Health. 11, 539-2458-11-539). The presence of ICD-10 anxiety- or affective disorder was ruled out in the controls after SCAN interviews (World Health Organization 1998). Blood samples from the bipolar cases were drawn in 2003/2004, from the depressed cases in 2007/2009 and from the controls in 2007. Informed consent was obtained from all donors in relation to study participation (Buttenschon, H. N. et al. 2010, Psychiatr. Genet. 20, 93-101; Kolstad, H. A. et al. 2011, Am. J. Epidemiol. 173, 94-102; Kaerlev, L. et al. 2011, BMC Public Health. 11, 539-2458-11-539).

Collection of Blood Samples and Isolation of Serum:

Blood for serum samples was collected in anticoagulant-free tubes (Terumo, Venosafe™, VF-109SP) and centrifuged (1550 g, 10 min, 4° C.) within 24 h. The supernatant was stored in aliquots at −80° C.

Quantification of serum sortilin levels: For quantification of sortilin, 96-well NuncMaxisorb F96 plates were coated with 10 μg/ml polyclonal rabbit anti-sortilin antibody (#5438) diluted in 100 mM NaHCO₃, pH 9.8 for 1 hour at 37° C. Coated wells were blocked with 2% bovine serum albumin (BSA) in PBS for 30 min at room temperature and washed four times in PBS containing 0.05% Tween-20. Dilution series of the purified extracellular domain of human sortilin were used for generating a standard curve. Samples, purified sortilin, primary and secondary antibodies were diluted in PBS containing 1% BSA and 0.05% Tween-20. Plates were incubated with samples for 1 hour at 37° C. and washed four times in PBS containing 0.05% Tween-20, and subsequently incubated with 1 μg/ml mouse monoclonal anti-sortilin (F11) (Gustafsen, C. et al. 2013, J. Neurosci. 33, 64-71) for 1 hour at 37° C. before another four times of washing. Finally, plates were incubated with HRP-conjugated rabbit anti-mouse IgG (Dako P0260) for 1 hour at RT and washed four times in PBS and four times in water before addition of o-phenylenediaminedihydrochloride/peroxide (OPD), followed by measurements of light absorbance at 490 nm using an ELISA plate reader.

Student's t-test was used for statistical analysis.

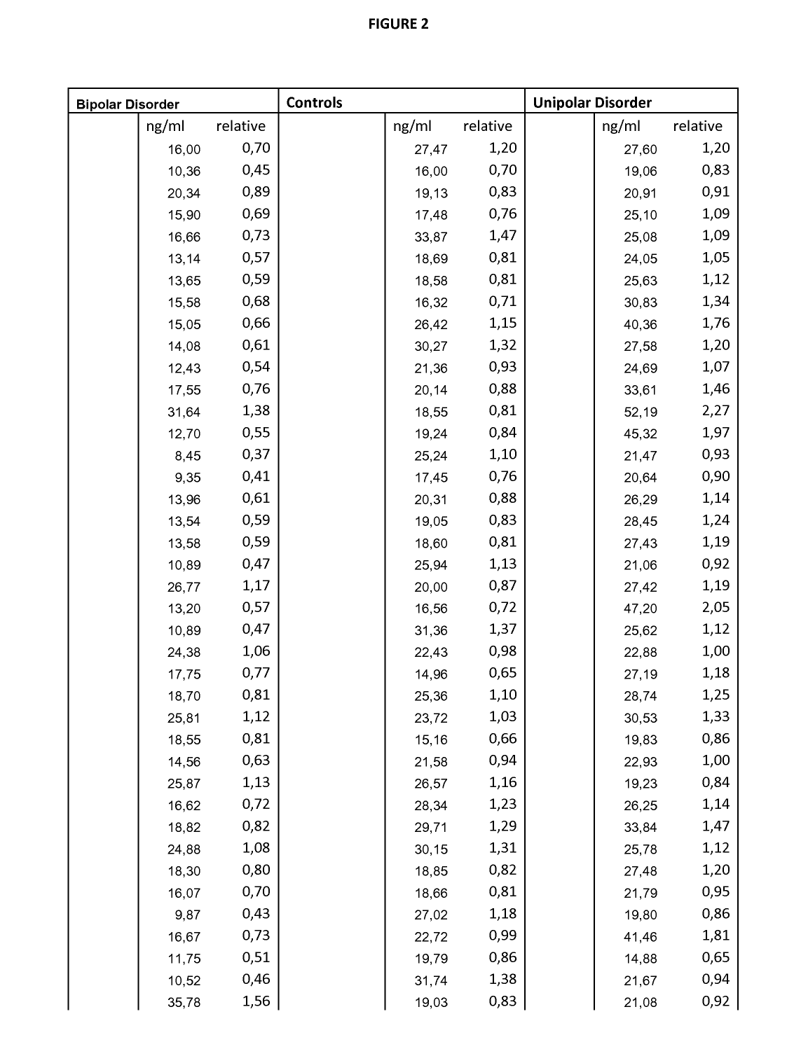

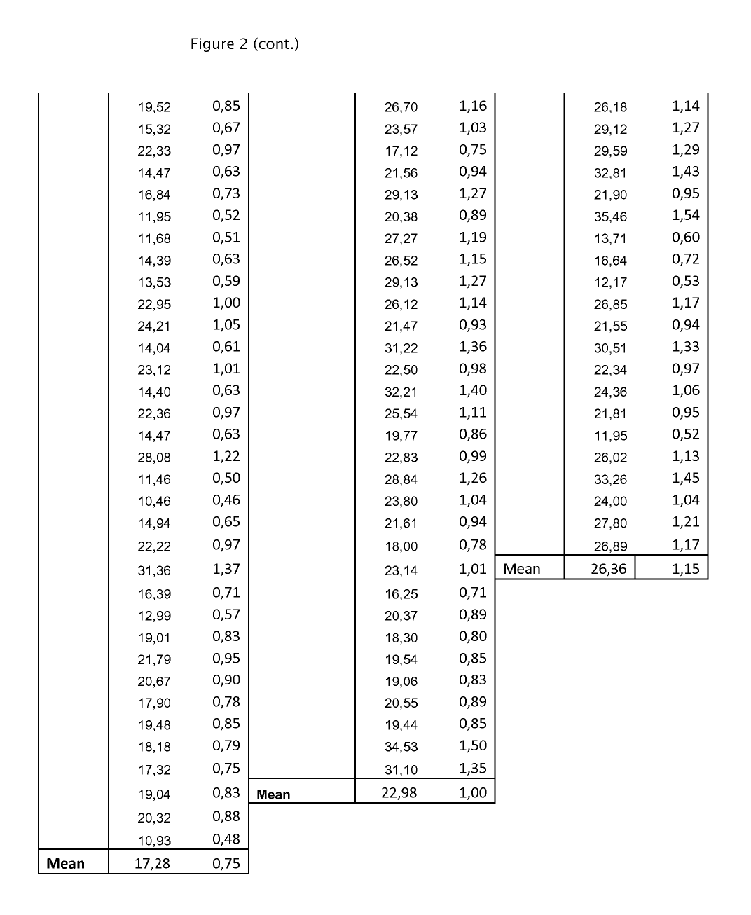

The results can be seen in FIGS. 1A, 1B and 2.

1. (canceled)

2. A method to diagnose or monitor bipolar or unipolar disorder in a subject, wherein said method comprises:
(a) causing a psychiatric examination of said subject to be conducted in order to diagnose the presence of such disorder or monitor its progression; and
(b) contacting a sample of tissue or fluid from said subject with an anti-Sortilin antibody under conditions sufficient to permit said anti-Sortilin antibody to immunospecifically and detectably bind said epitope of Sortilin if present in said sample at a concentration of between approximately 8.5 ng/ml and 47 ng/ml; and
(c) determining the concentration of said epitope of Sortilin in said sample by measuring said detected binding.

3. A method for treating a bipolar or unipolar disorder in a subject suffering from bipolar disorder, wherein said method comprises:
(a) causing a therapeutically effective amount of a mood-stabilizing agent or atypical antipsychotic to be administered to said subject;
(b) contacting sample of tissue or fluid from said subject with an anti-Sortilin antibody under conditions sufficient to permit said anti-Sortilin antibody to immunospecifically and detectably bind said epitope of Sortilin if present in said sample at a concentration of between approximately 8.5 ng/ml and 47 ng/ml; and
(c) determining the concentration of said epitope of Sortilin in said sample by measuring said detected binding.

4. An immunoassay kit for quantifying the level of Sortilin in a sample of tissue or fluid from a subject, wherein said kit comprises an anti-Sortilin antibody that is capable of immunospecifically binding an epitope of Sortilin (SEQ ID NO:1), and wherein said anti-Sortilin antibody is detectably-labeled, or said kit additionally comprises a detectable second antibody capable of immunospecifically binding a molecular complex of said anti-Sortilin antibody bound to said epitope of Sortilin, wherein said immunoassay is capable of detecting said epitope of Sortilin if present in said sample at a concentration of between approximately 8.5 ng/ml and 47 ng/ml.

5-6. (canceled)

7. The method according to claim 2, wherein said sample is a fluid sample.

8. The method according to claim 7, wherein said fluid sample is a blood, plasma or serum sample.

9-12. (canceled)

13. The method according to claim 3, wherein said mood-stabilizing agent or atypical antipsychotic is lithium carbonate, an anticonvulsant medicine or antipsychotic medicine, an antidepressant medicine, or a combination thereof.

14. The method according to claim 13, wherein said mood-stabilizing agent or atypical antipsychotic is an anticonvulsant medicine or antipsychotic medicine and is selected from the group consisting of: valproate, carbamazepine, lamotrigine, aripiprazole, olanzapine, quetiapine and risperidone.

15-26. (canceled)

27. The method according to claim 2, wherein said disorder is bipolar disorder.

28. The method according to claim 2, wherein said disorder is unipolar disorder.

29. The method according to claim 3, wherein said disorder is bipolar disorder.

30. The method according to claim 3, wherein said disorder is unipolar disorder.

31. The method according to claim 3, wherein said sample is a fluid sample.

32. The method according to claim 31, wherein said fluid sample is a blood, plasma or serum sample.

33. The immunoassay kit according to claim 4, wherein said anti-Sortilin antibody is detectably-labeled.

34. The immunoassay kit according to claim 4, wherein said kit comprises said detectable second antibody capable of immunospecifically binding a molecular complex of said anti-Sortilin antibody bound to said epitope of Sortilin.

35. The immunoassay kit according to claim 4, wherein said kit is capable of detecting said epitope of Sortilin in a fluid sample.

36. The immunoassay kit according to claim 4, wherein said fluid sample a blood, plasma or serum sample.


SEQUENCE LISTING (1 sequence)

SEQ ID NO: 1 - protein, 831 aa, Human
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE